(19) United States
(12) Patent Application Publication
(10) Pub. No.: US 2009/0283100 A1
(43) Pub. Date: Nov. 19, 2009

(54) ARTICLE AND METHOD FOR INDUCING PROPER BREATHING DURING SLEEP CYCLES TO REACTIVATE BODILY FUNCTIONS

(76) Inventor: Gene Hirs, Waterford, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/509,645

(22) Filed: Jul. 27, 2009

Related U.S. Application Data
(63) Continuation-in-part of application No. 12/135,773, filed on Jun. 9, 2008.
Application No. 12/135,773 is a continuation-in-part of application No. 12/043,737, filed on Mar. 6, 2008, now abandoned.
Application No. 12/043,737 is a continuation-in-part of application No. 11/465,342, filed on Aug. 17, 2006, now abandoned.

(51) Int. Cl. A61F 5/37

(52) U.S. Cl. 128/869

(57) ABSTRACT

All article and associated method for optimizing a person's sleep cycle (in particular an older individual's), particularly as it relates to the individual's ability to absorb increased amounts of oxygen by virtue of the associated device's construction to encourage breathing in nasally.

### CROSS-REFERENCE TO RELATED APPLICATIONS

This application is a continuation-in-part of U.S. patent application Ser. No. 12/135,773, filed Jun. 9, 2008, which is in turn a continuation-in-part of U.S. patent application Ser. No. 12/043,737 filed Mar. 6, 2008, which is a Continuation-in-part application of U.S. patent application Ser. No. 11/465,342, filed Aug. 17, 2006.

### FIELD OF THE INVENTION

The present inventions relate generally to articles and methods for inducing and maintaining proper sleep patterns in humans. In particular, the present invention teaches an article and associated method for optimizing a person's sleep cycle (in particular an older individual's), particularly as it relates to the individual's ability to absorb increased amounts of oxygen by virtue of the associated device's construction to encourage breathing in nasally.

### DESCRIPTION OF THE PRIOR ART

Incidences of improper breathing are becoming more commonplace as individuals age. Such irregular breathing cycles often are linked to physical and/or medical impediments as obesity, high blood pressure, arthritis, chronic fatigue, depression, infections, and sleep disturbances, such as sleep apnea.

A benefit of establishing proper breathing is in overcoming sleep apnea. This medically documented disturbance is created by the individual's tongue moving in a direction towards the back of the throat, thus resulting in a fatty tissue deposit at that region being pushed over the windpipe. As a result, the individual is caused to choke, typically resulting in the person gasping and/or waking. In rare instances, coma or death can result from a severe apnea tic episode.

The accepted current practice of overcoming sleep apnea is by having the person breathe only through the nose. The mouth is shut with a chin strap to prevent mouth breathing. A low pressure air blower supplies air to the nose, for inhaling. Exhaling pressure is higher than the blower pressure and can exit from the nose. In cases where the nose malfunctions the blower air enters the mouth and the exhaust overcomes the blower pressure and air exits from the mouth. This is a rare occurrence but a few need this technique.

Portnoy, U.S. Pat. No. 6,089,232, teaches a method and device for preventing snoring and which teaches an elastic panel having an adhesive backing and a narrow horizontal slit cut therein. The panel is applied over the mouth and upper and lower lips of the person tightly enough to prevent the person's mouth from opening during sleep.

Patent Publication No. 2007/011948 involves a method of wearing a filtering face mask with a flapper valve with a low pressure blower which you must transport wherever you go. The 948'reference also does not prevent mouth breathing to occur and may require chin straps to properly function.

JP 1U4081A involves a flap for exhaling from the mouth, this creating a tongue movement that causes apnea. JP 2004/34431 involves a mouthpiece held by the teeth. In sleep it is impossible to bite down to control an object resulting in mouth breathing. Accordingly, the above methods are cumbersome and do not result in an answer to the sleep apnea problem.

To overcome sleep apnea, for the majority, the breathing must be through the nose. This results in eliminating any tongue movement and no blocking of the windpipe. Therefore the mouth remains shut during the entire sleep period, this method must be learned because most of the people with sleep apnea are mouth breathers. This is accomplished with an assist article for establishing proper breathing patterns and which includes a tape adapted to be resealably secured upon the mouth of a person including a central aperture into which the tip of the lips are placed. The tape forces the person to breathe through the nose and receives a steady supply of air for proper breathing. The subconscious mind then does not get signals that the person is starving from lack of air and therefore does not disturb a person with sleep interruptions and apnea.

### SUMMARY OF THE INVENTION

An assist article for establishing proper sleeping patterns including a tape adapted to be releasably secured to the outside of the lower and upper lips of a person and including a circular inner aperture defined by an inner edge extending and rim surface, through which the tips of the lips of a person are capable of passing in extended and projecting fashion. The tape establishes a path for air flow so as to result in breathing only through the person's nasal passages, the tape preventing tongue movement which allows the epiglottis to remain open and the air free access to the windpipe, maximizing breathing volume, this in contrast to Portnoy in which the narrow slot shaped aperture defined in the elastic article prevents the user from fully seating the lips through the narrow slot configuration.

The object of this invention is for a permanent cure of apnea, proving that sleep can be achieved apnea-free after about six months without any assist article and by establishing consistent sleeping pattern of the individual with motionless lips and tongue during sleep, through the use of the present device.

### BRIEF DESCRIPTION OF THE DRAWINGS

Reference will now be made to the attached drawings, when read in combination with the following description, wherein like reference numerals refer to like parts throughout the several views, and in which:

FIG. 1 is a side profile view illustrating the device according to the present inventions in an installed configuration and by which the user's lips are compressed, resulting in a nasal directed intake of air;

FIG. 2 is a diagram of oxygen saturation in the blood with and without the mouth tape;

FIG. 3 is a plan view of the breath assist device according to the inventions and which includes a flexible and impermeable one-sided adhesive body, combined with a centrally disposed (lip seating) aperture; and

FIG. 4 is an illustration of an oximetry (oxygen in blood) concentration of a non-wearer over a ten day period.

### DETAILED DESCRIPTION OF THE PREFERRED EMBODIMENTS

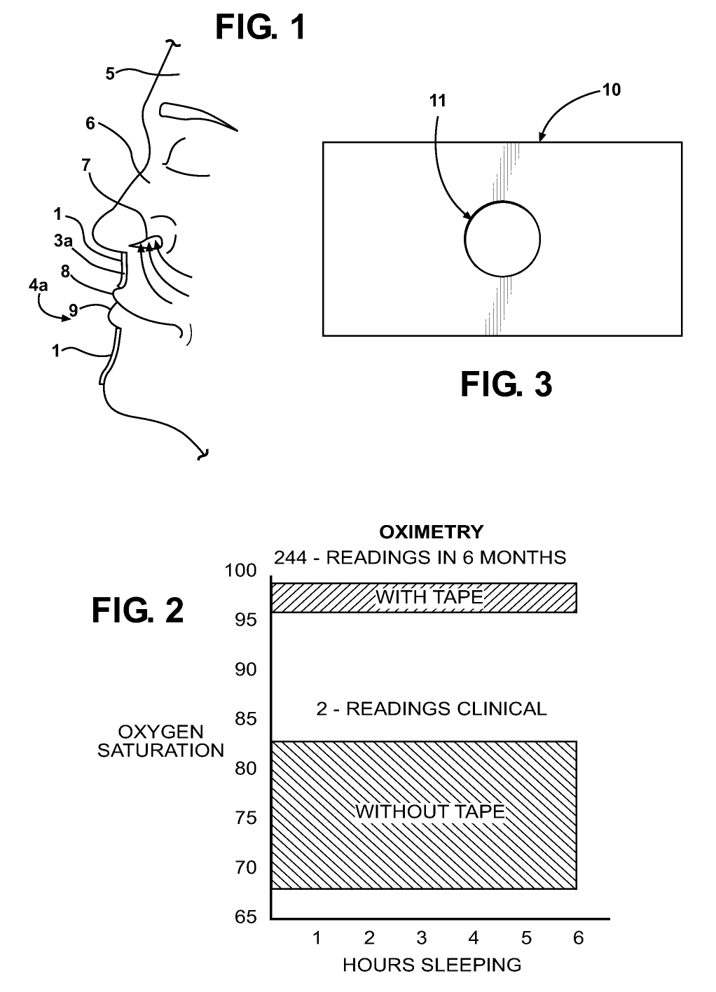

Referring now to FIG. 1-3, in combination, an article and associated method is now described for assisting in maintaining proper breathing cycles of a person according to the present inventions. As previously described, the article and method of the present invention operates to optimize a person's sleep cycle (in particular an older individual's), particularly as it relates to the individual's ability to absorb increased amounts of oxygen by virtue of the associated device's construction to encourage breathing in nasally (through the nose).

The breathing assist device, as best shown in FIG. 3, includes a flexible and impermeable one-sided adhesive body 10. The body is constructed from such as a tape having a rectangular shape and a centrally disposed (lip seating) aperture 12, defined by an inner closed rim surface, and which is sized in an appropriate fashion (such as in one instance between 4″in length by 2″in height) and defines in inner continuous annular or circular edge within the body 9, the inner circular profile or opening in one instance being ¾″ in diameter.

FIG. 1 further illustrates, at 4a, a side profile outline of the device according to the present invention, corresponding to the body 10 described in FIG. 3 and including applied locations 1 upon a user's face 5 and which includes, in relevant part, a nose 6 with internal nasal passages (of which a single one 7 is illustrated in side profile) and a mouth defined by upper and lower lips 8 and 9. In FIG. 1, the user's lips are compressed upon being pre-located through the aperture and in contacting fashion with the inner defined rim, owing to the substantially closed position associated with the compressing edge 3a of the inner aperture, thus resulting in a nasal breathing as a result of the tape preventing mouth and tongue movement, further allowing the wearer's epiglottis to remain open during sleep and resulting in free access of air to the wind pipe, maximizing breathing volume.

FIG. 2 is a diagrammatic illustration of oxygen saturation during six hours of sleeping time. According to experimental data obtained, two sleeping periods were administered in a hospital test environment, and without the device. There were also 244 readings with a finger inserted oximeter administered at home with the tape.

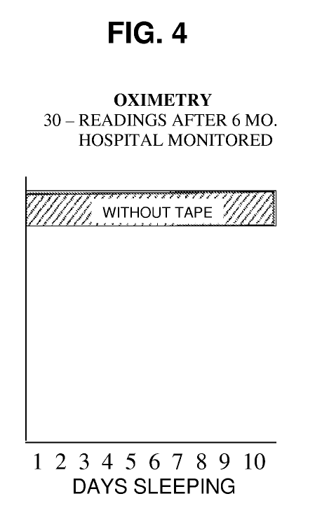

FIG. 4 indicates an illustration of an oximetry (oxygen in blood) concentration of a non-wearer over a ten day period. This in particular indicates the monitoring of vital signs (SPO²) which are representative of oxygen concentration in the blood. As is known, a person with apnea does not receive enough oxygen because of air flow restriction. Curing this malady can be accomplished with repeated wearing of the article of the present invention, combined with discipline on the part of the wearer. The chart further represents a ten day period of monitoring oxygen levels by the hospital or other record keeping authority. Approximately six readings are taken every day, including three during daytime hours and three during a given sleep cycle.

A method for preventing apnea further includes the steps of maintaining an oxygen concentration level in the blood of 97% to 100% during sleep, such as by nasal breathing as a result of wearing the tape over a number of repetitive cycles, as well as after discarding the tape following successful subconscious and repetitive retraining of the wearer to prevent mouth and tongue movement, further allowing the wearer's epiglottis to remain open during sleep and resulting in free access of air to the wind pipe, maximizing breathing volume.

Concurrently the patients windpipe is left open by restricting tongue movement, this resulting from the application of the tape outside of the lower and upper lips with the central define hole, for inserting (sending) therethrough the patient's upper and lower lips.

Having described my invention, other and additional preferred embodiments will become apparent to those skilled in the art to which it pertains, and without deviating from the scope of the appended claims.

I claim:

1. An article for inducing a wearer to breathe nasally during a sleep cycle for curing sleep apnea, comprising:
a non-elastic one sided tape applied to an outside of a wearer's mouth, the tape exhibiting a circular aperture defined by an inner rim which is of sufficient size to permit the user's lips to locate through and protrude beyond the rim in order to subconsciously and repetitively train the wearer to prevent mouth and tongue movement, further allowing the wearer's epiglottis to remain open during sleep and resulting in free access of air to the wind pipe, maximizing breathing volume.

2. The article as described in claim 1, further comprising said tape exhibiting a rectangular shape 4″long by 2″high with ¾″ dia. central opening.

3. A method for repetitively instructing a wearer to breathe nasally during sleep cycles, comprising the steps of:
forming a circular shaped aperture within a non-elastic and planar shaped tape exhibiting a one-sided adhesive;
applying the tape to an outside of the wearer's mouth, causing the lips to locate through and protrude beyond an inner defined rim of the aperture;
training the sub-conscious mind of the wearer in a habit forming fashion to prevent mouth and tongue movement, further allowing the wearer's epiglottis to remain open during sleep and resulting in free access of air to the wind pipe, maximizing breathing volume; and
discarding the tape upon establishing a sufficient retraining of the wearer to achieve uninterrupted sleep.

4. The method as described in claim 3, fiber comprising the step of achieving and maintaining an oxygen saturation level in a range of 97% to 100% within the wearer's bloodstream after wearing the tape over a number of repetitive cycles as well as after discarding the tape.
